(19) United States
(12) Patent Application Publication
(10) Pub. No.: US 2026/0263811 A1
(43) Pub. Date: Sep. 10, 2026

(54) Neuromodulatory Method for Treating Neurological Disorders

(71) Applicant: Dirk De Ridder, Dunedin (NZ)

(72) Inventor: Dirk De Ridder, Dunedin (NZ)

(21) Appl. No.: 17/209,142

(22) Filed: Mar. 22, 2021

Related U.S. Application Data
(60) Provisional application No. 62/992,615, filed on Mar. 20, 2020.

(51) Int. Cl. A61N 1/36

(52) U.S. Cl. CPC A61N 1/36171; A61N 1/3616; A61N 1/36178

(57) ABSTRACT

A method for the treatment of disorders of the nervous system, in which one or more target nerves receive electrical stimulation signals that combine one or more frequency spectrums having a power spectral density per unit of bandwidth proportional to 1/f^{(-β)} noise and of one or more frequency spectrums having a power spectral density per unit of bandwidth proportional to 1/f^{β} noise, optionally combined with a regimen of applying such stimulation in a tonic manner or in a burst manner.

### RELATED APPLICATION

This application claims the benefit of U.S. Provisional Application No. 62/992,615 filed Mar. 20, 2020, which is incorporated herein by reference in its entirety.

### BACKGROUND OF THE INVENTION

The present invention relates to a method of using an IMD with a pulse generator, the method of which can be utilized to treat neurological conditions and/or disorders. More particularly, and not by way of limitation, the present invention is directed to a method for using 1/f^{(-β)} and 1/f^{β} noise together or sequentially to treat neurological conditions and/or disorders.

### TECHNICAL FIELD OF THE INVENTION

Different firing modes or frequencies occur in the brain and/or other neuronal tissue, for example tonic firing and burst firing (irregular or regular burst firing). Such firing modes can be utilized for normal processing of information, however, alteration of the firing modes, may also lead to pathology.

For example, certain neurological conditions are associated with hyperactivity of the brain and can be traced to a rhythmic burst firing or high frequency tonic firing or hypersynchronous firing (e.g., tinnitus, pain, depression, tremor, Parkinson's disease and epilepsy).

During the past decade, neuromodulation systems have been used to modulate various areas of the brain, spinal cord, or peripheral nerves (See, for example, U.S. Pat. Nos. 6,671,555; 6,690,974, the entire disclosure of which are incorporated herein by reference). These types of systems utilize tonic forms of electrical stimulation.

Recently the brain and its function has been conceived as a Bayesian prediction machine. This concept of brain functioning states that the brain predicts upcoming events based on prior beliefs (stored in memory) and updates these prior beliefs by sampling the environment via the senses. The prior belief that best fits the sensed environmental input survives and becomes the posterior belief, i.e. a percept, a thought, an idea, action or behavior. If there is no prediction error the posterior believe equals the prior belief and a status quo is maintained. there is a prediction error the brain will attend to this prediction error. The predictions (prior beliefs) are generated in the beta frequency band and the prediction errors encoded in the gamma frequency band. Many pathologies are characterized by persisting prediction errors and or abnormal prior beliefs, i.e. by abnormal gamma and beta band activity respectively. These pathologies include, but are not limited pain, tinnitus, anxiety and depression, epilepsy, Parkinson disease, tremor, dystonia, addiction (food, alcohol, sex, gambling, illegal drugs), obsessive compulsive disorder, ADHD, Alzheimer and other dementias, personality disorders etc. The pathologies associated with abnormal predictions or persistent prediction errors include neurological, psychological and psychiatric disorders, as this is a general mechanism involved in those pathologies. The present invention is the first to apply a combination of 1/f^{(-β)} noise and 1/f^{(β)} noise to achieve stimulation of tissues in order to treat a neurological, psychological, or psychiatric pathology condition.

### BRIEF SUMMARY OF THE INVENTION

The present invention includes a method of stimulating nerve tissue of a patient using an IMD, the method comprising generating, by an IMD containing a pulse generator, a stimulus that comprises a signal that is produced from a frequency spectrum having a power spectral density per unit of bandwidth proportional to 1/f^{β} and 1/f^{(-β)}, i.e. combined noise stimulation (CNS), wherein β is a real number but that excludes 0; providing the stimulus from the IMD containing a pulse generator to at least one stimulation lead; and applying the stimulus to nerve tissue of the patient via one or several electrodes of at least one stimulation lead. A variation of this embodiment has the stimulus combined with at least one pulse stimulus to be repeated in a tonic manner or combined with a burst stimulus.

Another embodiment of this invention includes a method of stimulating nerve tissue of a patient using an IMD containing a pulse generator, the method comprising storing, in the IMD containing a pulse generator, one first stimulation parameter that defines a frequency to be used as the lower bound of a frequency spectrum; storing, in the IMD containing a pulse generator, one second stimulation parameter that defines a frequency to be used as the upper bound of a frequency spectrum; generating, by IMD containing a pulse generator, a stimulus that comprises a frequency spectrum between the first stimulation parameter and second stimulation parameter, wherein the frequency and power of the frequency spectrum are inversely proportional up to a certain frequency and proportional above that frequency; providing the stimulus from the implantable IMD containing a pulse generator to at least one stimulation lead; and applying the stimulus to nerve tissue of the patient via at least one electrode of at least one stimulation lead.

Another embodiment of this invention includes a method of stimulating nerve tissue of a patient using an IMD containing a pulse generator, the method comprising storing, in the IMD containing a pulse generator, one first stimulation parameter that defines a frequency to be used as the lower bound of a frequency spectrum; storing, in the IMD containing a pulse generator, one second stimulation parameter that defines a frequency to be used as the upper bound of a frequency spectrum; storing, in IMD containing a pulse generator, one third stimulation parameter that defines a frequency at which a peak of a pre-determined amplitude is to occur; generating, by IMD containing a pulse generator, a stimulus that comprises a frequency spectrum between the first stimulation parameter and second stimulation parameter, wherein the power spectral density is inversely proportional to a specific frequency and proportional above that frequency, wherein a peak of a pre-determined amplitude occurs at the frequency defined by the third stimulation parameter; providing the stimulus from the IMD containing a pulse generator to at least one stimulation lead; and applying the stimulus to nerve tissue of the patient via one or several electrodes of at least one stimulation lead.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In the figures, elements having the same designation have the same or similar functions.

FIGS. 1A through 1D illustrate 1/f^{β} noise. FIG. 1A shows an exemplary 1/f^{β} noise spectrum generated by a power source where B has a value of 1. FIG. 1B shows an exemplary 1/f^{β} noise spectrum where β has a value of 1. FIG. 1C shows 1/f^{β} noise spectrum generated by a power source where β has a value of 2. FIG. 1D shows an exemplary 1/f^{β} noise spectrum where β has a value of 2.

FIGS. 2A and 2B illustrate 1/f^{β} noise where β has a value of zero. FIG. 2A shows an exemplary 1/f^{β} noise spectrum generated by a power source where β has a value of 0. FIG. 2B shows an exemplary 1/f^{β} noise spectrum where β has a value of 0.

FIGS. 3A through 3D illustrate 1/f^{(-β)} noise. FIG. 3A shows an exemplary 1/f^{(-β)} noise spectrum generated by a power source where β has a value of 1. FIG. 3B shows an exemplary 1/f^{(-β)} noise spectrum where β has a value of 1. FIG. 3C shows 1/f^{(-β)} noise spectrum generated by a power source where β has a value of 2. FIG. 3D shows an exemplary 1/f^{(-β)} noise spectrum where β has a value of 2.

FIG. 4 illustrates composite noise stimulation, noise spectra, and component parts of composite noise stimulation that vary where values of β are equal to 1 or 2.

FIG. 4 at 401, 403, and 405 illustrate composite noise stimulation, 1/f^{β} and 1/f^{(-β)}. FIG. 4 at 405 shows an exemplary 1/f^{β} and 1/f^{(-β)} noise spectrum where β has a value of 1 (the flatter parabola) and where β has a value of 2 (the steeper parabola). FIG. 4 at 403 shows 1/f^{β} and 1/f^{(-β)} noise spectrum generated by a power source where β has a value of 1. FIG. 4 at 417, 419, 421, 423, and 425 shows the component parts 1/f^{β} and 1/f^{(-β)} as separate noise generated by a power source where β has a value of 1 and shows the composite noise spectrum where β has a value of 1. FIG. 4 at 407, 409, 411, 413, and 415 shows an exemplary 1/f^{(-β)} noise spectrum where β has a value of 2.

FIG. 4 at 417, 419, 421, 423 and 424 illustrate composite noise stimulation, 1/f^{β} and 1/f^{(-β)} where β has a value of 1. FIG. 4 at 417 shows an exemplary 1/f^{(-β)} noise spectrum generated by a power source where β has a value of 1. FIG. 4 at 417 shows an exemplary 1/f^{β} noise spectrum generated by a power source where β has a value of 1. FIG. 4 at 421 shows an exemplary 1/f^{(-β)} noise spectrum where β has a value of 1. FIG. 4 at 419 shows an exemplary 1/f^{β} noise spectrum where β has a value of 1. FIG. 4 at 425 shows an exemplary composite noise stimulation, 1/f^{β} and 1/f^{(-β)}, noise spectrum where β has a value of 1.

FIG. 4 at 407 through 409, 411, and 413 illustrate composite noise stimulation, 1/f^{β} and 1/f^{(-β)} where β has a value of 2. FIG. 4 at 411 and 413 shows an exemplary 1/f^{(-β)} noise spectrum generated by a power source where β has a value of 2. FIG. 4 at 409 shows an exemplary 1/f^{β} noise spectrum generated by a power source where β has a value of 2. FIG. 4 at 413 shows an exemplary 1/f^{(-β)} noise spectrum where β has a value of 2. FIG. 4 at 411 shows an exemplary 1/f^{β} noise spectrum where β has a value of 2. FIG. 4 at 407 shows an exemplary composite noise stimulation, 1/f^{β} and 1/f^{(-β)}, noise spectrum where β has a value of 2.

FIGS. 5A-5J illustrate examples of electrical stimulation leads that may be used to electrically stimulate neuronal tissue.

FIG. 6 depicts a stimulation system that can measure or detect given neuron signals that can be used to modulate the 1/f^{β} and 1/f^{(-β)} noise stimulation according to one representative embodiment.

FIG. 7 is an example of a system of electrical stimulation leads that may be used to electrically stimulate neuronal tissue.

FIG. 8 depicts a stimulation system that can sense and/or monitor sleep stage that can be used to alter therapy.

FIG. 9 shows modules within the memory of FIG. 8.

### DETAILED DESCRIPTION

The method and system described herein relate to stimulating nerve tissue to treat a neurological disease and/or condition. Using an IMD containing a pulse generator, a stimulus is created that comprises a noisy signal. The noisy signal is produced from a composition of noisy signals where each of the more than one component of the composition contains a different frequency spectrum.

The brain functions and is structured as following 1/f^{β} noise. The spectral exponent β is typically close to 1 when brain function is measured in the awake state with electroencephalography (EEG), magnetoencephalography (MEG) or functional magnetic resonance imaging (fMRI). However, when recording directly from the brain with local field potentials or electrocorticography (ECOG) the β is typically closer to 2. When the level of consciousness decreases, as in sleep, anesthesia or coma the β exponent also increases.

The method and system described herein consists of combining 1/f^{β} noise and 1/f^{(-β)} noise stimulation. This combines the benefits of 1/f^{β} noise and 1/f^{(-β)} noise. 1/f^{β} noise permits flexible stability by mimicking the naturally occurring stability of brain, spine, ganglion or nerve activity and the 1/f^{(-β)} noise permits to specifically target pathological beta and gamma activity reflective of predictions and prediction errors. Thus, this composite noise stimulation on the one hand mimics and thereby strengthens naturally ongoing healthy brain activity and on the other hand specifically targets and disrupts pathological beta and gamma activity associated with disorders of the nervous system.

The method and system described herein relate to stimulating nerve tissue to treat a neurological disease and/or condition. Using an IMD containing a pulse generator, a stimulus is created that comprises a signal that is produced from a frequency spectrum having a power spectral density per unit of bandwidth proportional to 1/f^{β} for the low frequencies and 1/f^{(-β)} for the high frequencies, wherein β excludes 0. β can be any real number, including natural, integer, rational, irrational, complex or fluctuating number. For example, β=1 or β=2, but as stated, it can also be irrational numbers such as π, e, or φ. A real number is a value of a continuous quantity that can represent a distance along a line. Real numbers include all the rational numbers, such as the integer −5 and the fraction 4/3, and all the irrational numbers, such as √2 (1.41421356 . . . , the square root of 2, an irrational algebraic number). Included within the irrationals are the transcendental numbers, such as π (3.14159265 . . . ). The stimulus is provided from the IMD containing a pulse generator to at least one stimulation lead; and applied to nerve tissue of the patient via one or several electrodes of at least one stimulation lead.

Yet further, the stimulus can be combined with at least one pulse stimulus repeated in a tonic manner or a burst stimulus that comprises a plurality of groups of spike pulses.

Still further, the stimulus can be modulated at any specific frequency, either by selective power increase, envelope modulation or adding more tonic or burst stimuli of this frequency.

A first stimulation parameter that defines a frequency having a lower bound of a frequency spectrum (0.01 Hz) and a second stimulation that defines a frequency having an upper bound of a frequency spectrum (1000 Hz) can be stored in a controller or IMD containing a pulse generator and such controller can be used to generate a stimulus that comprises a frequency spectrum between the first and second stimulation parameters such that the frequency and power of the frequency spectrum are inversely proportional up to a certain frequency and proportional above that frequency.

The composite noise stimulation that combines 1/f^{β} for the low frequencies and 1/f^{(-β)} for the high frequencies creates a trough which can be selected broadly to be between 1 and 40 Hz, but can typically be at 8-12 Hz for thalamocortical stimulation, 4-7 Hz for memory and emotion circuit stimulation, or 1-3 Hz for reward and autonomic circuit stimulation. This trough can also be selected based on the individual alpha peak frequency.

The lower part of the noise stimulation can start with a plateau, and the higher part of the stimulation can level off with a plateau, so that the amplitude in the low and high frequencies does not become excessive. This means that the β of the low frequencies, i.e. the 1/f^{β} component of the noise is near to but not at 0 at the lower end of the frequency domain, and that the −β of the high frequencies, i.e. the 1/f^{-β} component approaches but does not equal 0 such as at the higher frequencies. This leveling off at low stimulation frequencies can typically be between 0.01 and 0.1 Hz, but also at 0.5 or 1 Hz. The leveling off at high frequencies can typically be at 40-100 Hz.

The slope of the 1/f^{β} can be symmetrical to 1/f^{(-β)}, i.e. the values of β and −β can be the same, but in another embodiment the values can differ.

Currently, stimulation generated by IMDs are not physiologically similar to the endogenous electrical signals generated by the brain. The exogenous electrical signals generated by the IMDs may result in epileptic events and/or in brain habituation to stimulation signals over time. The inventor is the first to describe a composition stimulation design composed of parameters in which a 1/f^{(-β)} (one over frequency raised to the power of minus β) noise and 1/f^{β} (one over frequency raised to the power of β) noise is used to disrupt abnormal brain wave frequency band activity to treat a neurological condition.

The following section more generally describes an example of a procedure for treatment using a composite of 1/f^{(-β)} noise and 1/f^{β} noise. A stimulation paradigm that is a composite of 1/f^{(-β)} noise and 1/f^{β} noise is known herein as “Composite Noise Stimulation” noise (CNS).

1/f^{(-β)} noise stimulation is described in more detail in the inventor's co-pending application U.S. Nonprovisional patent Ser. No. 17/209,149, filed Mar. 22, 2021, which is incorporated by reference herein in full. Those skilled in the art will recognize that the choice of 1/f^{(-β)} noise stimulation alone, or as part of CNS depends on the goals of the treatment paradigm, the patient and the outcome desired, and does not change the invention described herein.

1/f^{β} noise stimulation is described in more detail in U.S. patent application Ser. No. 12/221,548, which is incorporated by reference herein in full. Those skilled in the art will recognize that the choice of 1/f^{β} noise stimulation alone, or as part of CNS depends on the goals of the treatment paradigm, the patient and the outcome desired, and does not change the invention described herein.

CNS allows for the optimization of many parameters that provide certain effects including (without limitation) the following: a set and/or range of stimulation waveforms and/or protocols that can completely eliminate neurological disease/disorder; a set and/or range of efficacious stimulation waveforms and/or protocols that requires the lowest voltage; and a waveform and/or protocol that maintains treatment efficacy over long periods of time such as a waveform and/or protocol that can prevent habituation or adaptation, and a waveform and/or protocol that is anti-epileptic.

Still further, the generated CNS signal can be, for example, filtered, combined, or otherwise processed whereby the generated CNS is utilized as a background noise signal over another signal with a spectral peak at a selected frequency. For example, the spectral peak can be an alpha peak, beta peak, delta peak and/or theta peak. Any one or more of these peaks can be added to the 1/f^{(-β)} and/or the 1/f^{β} noise. The peaks can be generated using typical known frequencies or the peaks can be individualized for each patient.

Yet further, the CNS can be combined with standard tonic and/or burst stimulation to further enhance the optimization or prevent habituation. Combinations of tonic and/or burst stimulation are known in the art, for example, U.S. Pat. Nos. 7,734,340 and 8,364,273, which are incorporated by reference in their entirety.

The predetermined site for stimulation can include, for example, peripheral neuronal tissue and/or central neuronal tissue. Peripheral neuronal tissue can include a nerve root or root ganglion, or any peripheral neuronal tissue associated with a given dermatome or any neuronal tissue that lies outside the central neuronal tissue such as the brain, brainstem or spinal cord.

### 1. Composite Noise Stimulation

A noise signal can be described as a signal that is generated according to a random process. In practice, various algorithms (e.g., in software executed on a processor) are employed to simulate a given random process to generate a “pseudo-random” signal where the generated pseudo-random signal possesses similar characteristics with signals corresponding and correlated to a random process. The characteristics of a particular noise signal depend upon the underlying process generating the noise signal. For example, the power spectral density or power distribution in the frequency domain may be employed to characterize the random process and, hence, also characterize a corresponding time-domain noise signal. The classification of the power spectral density of a noise signal may be described in reference to noise signal's algorithm associated with different types of power spectral densities.

According to these conventions, the power spectral density of CNS can be defined herein as composition of the sequential application of 1/f^{(-β)} noise and then 1/f^{β} noise. For these equations, f represents frequency and β is a value selected to characterize the noise signal.

CNS has advantages over current stimulation paradigms, because of CNS′ ability to disrupt pathological activity. These advantages arise due to the unique features of sequentially combining 1/f^{β} noise with 1/f^{(-β)} noise. These advantages include permitting stability and permitting adaptive flexibility via stochastic resonance effect due to the 1/f^{β} component, which mimics naturally and physiologically occurring activity in the brain. The 1/f^{(-β)} component permits drowning rhythmic pathological signals in beta and gamma and permitting better analogue to digital conversion at receptor site.

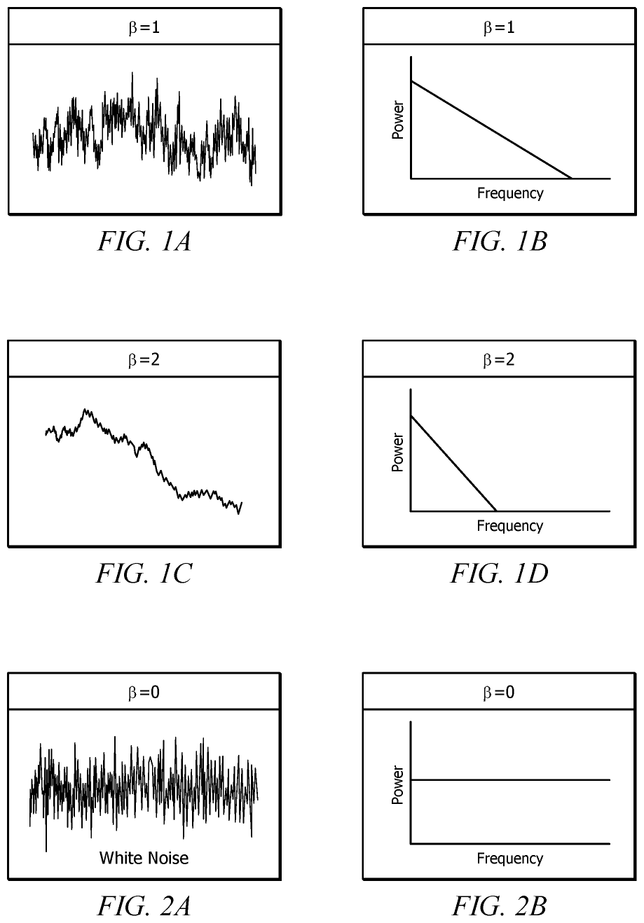

When taken separately as 1/f^{β} noise and 1/f^{(-β)} noise, the two components are inversely proportional to each other when β is the same real number for both equations. FIGS. 1A and 1B shows 1/f^{β} when β=1, while FIGS. 1C and 1D shows 1/f^{β} when β=2. As can be seen in comparing FIGS. 1B and 1D, as β increases, the slope of 1/f^{β} increases.

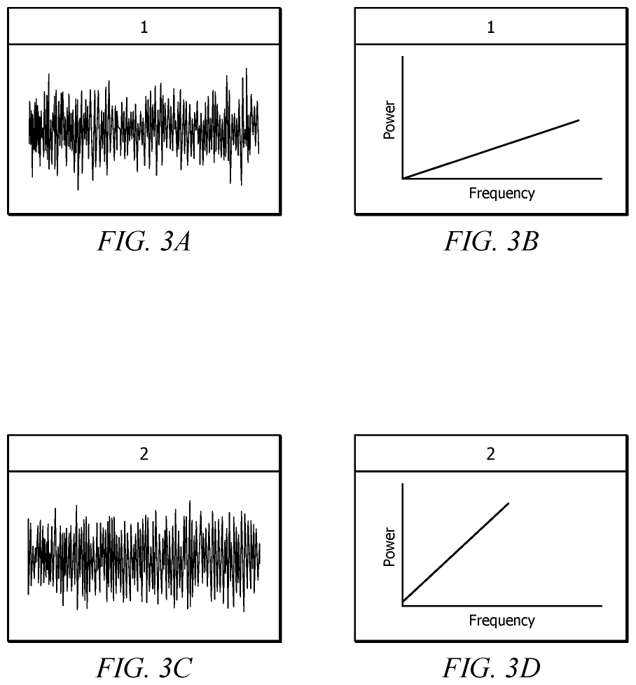

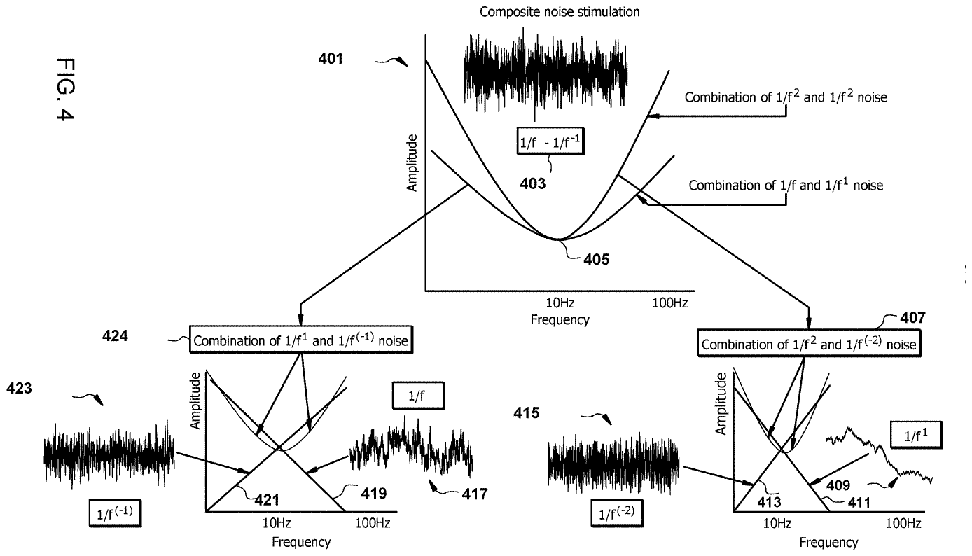

FIG. 3 shows the 1/f^{(-β)} relationships as β changes to a higher real number. FIGS. 3A and 3B shows the 1/f^{(-β)} when β=1, while FIG. 3C and FIG. 3D shows 1/f^{(-β)} when β=2. As can be seen in comparing FIGS. 3B and 3D, as β increases, the slope of 1/f^{β} increases.

FIG. 4 at 401 shows 1/f^{(β)} and 1/f^{(-β)} in a graphical representation. For CNS, where β for each component has the value of 1 is shown in FIG. 4 at 425. FIG. 4 at 425 shows the representation of CNS where β for each component equals 1. As can be seen in FIG. 4 at 401, 407, and 425, CNS, when plotted on an amplitude versus power graph has a parabolic shape. Again, as shown in FIG. 4 at 401, the parabolic shape of CNS becomes shallower as β approaches zero. As shown in FIG. 4 at 425 the parabolic shape of CNS becomes steeper as β increases in its absolute value.

In a different embodiment, the value of β can be one value for the first component of CNS comprising 1/f^{β} and a different value for the second component of the CNS comprising 1/f^{β)}. Whether it's the same for both components of CNS or different for each component, β can be, for example, any real, natural, integer, rational, irrational or complex number. For example, the spectral density for white noise is flat (β=0). Alternatively, any portion (which includes where both portions have the same value or each portion individually has a different value) of CNS B can equal 1, 2 or it can be greater than 2 as shown in FIG. 2B (e.g. 3, 4, 5 . . . etc.). Suitable non-integer β values about 1 include 0.5, 0.6, 0.7, 0.8, 0.9, 1.0, 1.1, 1.2, 1.3, 1.5, or any values there between for some embodiments. Likewise, suitable non-integer β values about 2 can include 1.5, 1.6, 1.7, 1.8, 1.9, 2.0, 2.1, 2.2, 2.3, 2.4, 2.5 or any value there between for some embodiments. In general, for non-integer values about an integer X, β values can include Y.5, Y.6, Y.7, Y.8, Y.9, X, X.1, X.2, X.3, X.4, X.5, where Y equals X minus 1.

Abnormal electrical and/or neural activity is associated with different diseases and disorders in the central and peripheral nervous systems. In addition to a drug regimen or surgical intervention, potential treatments for such diseases and disorders include using a stimulating system that includes an implantable medical device (IMD) for electrical stimulation of a portion or all of the patient's body tissue. For example, the IMD that provides the electrical stimulation can be an implantable IMD containing an implantable pulse generator (IPG) or a radio frequency pulse generator (RF). Those skilled in the art know that both IPG and RF systems contain internal pulse generators for stimulating nervous tissue. One difference between an IPG and a RF system is the location of each system's power source. An IPG's power source is usually a battery and is contained within the IPG, which is implanted in the patient's body. A RF system's power source is external to the human body, such that power is transmitted using radio frequency waves that are inductively linked to a radio frequency antennae or receiver coupled to or in the RF device, which is located in the human body. An example IPG system is the Algovita system formerly marketed by Nuvectra®. An example RF system is Nalu®'s neurostimulation system. In particular, an IMD may electrically stimulate a target neuronal tissue location by the selective application of controlled electrical input signals to one or more electrodes coupled to or placed in proximity to the patient's neuronal tissue. Such electrical input signals may be applied to the patient's neuronal tissue in order to treat a neurological disease, condition, or disorder.

The response of nonlinear systems to signal (normal or abnormal) may be optimized or treated by combining the signal with a non-negligible level of noise. This optimization or increased sensitivity cause by a noisy (stochastic) signal is referred to herein as “Stochastic Resonance.” Said another way, when a noisy (stochastic) signal is intentionally injected or added into a nonlinear (for example, neurological) system, the system's sensitivity increases. This optimization or increased sensitivity is also called “dithering.” Dithering is a form of stochastic resonance.

A specific example of dithering is CNS. By adding a composite signal sequentially composed of a first 1/f^{(-β)} component and second 1/f^{β} component, a nervous system's ability to process or treat an abnormal or weak signal created by the nervous system is enhanced. In this dithering example, the nervous system becomes more sensitive or optimized due to the CNS. This optimization or increased sensitivity allows the nervous system to process the abnormal or weak signal in a way that today's neuromodulation signals do not. Dithering improves the brain's ability to correctly process weak or abnormal signals corresponding to certain diseases states from a normal signal. Non-limiting examples of disease states that have weak or abnormal signals are chronic pain, tinnitus, Parkinson disease, depression, anxiety, paresthesia, addiction, OCD and others. Those skilled in the art understand that the previous examples of disease states are not meant to be limiting examples and that treatment using a CNS signal will apply to disease states that contains weak or abnormal signals.

For a system to exhibit stochastic resonance there needs to be a threshold that must be exceeded in order to activate the system. When the input signal is not strong enough to exceed a threshold, noise, even if in small amounts, added either to the system or the signal may occasionally suffice to trigger activation. Typically, this type of phenomenon is associated with 1/f^{(-β)} noise, where β equals zero, which is sometimes referred to as “pure noise” or “white noise” as shown in FIGS. 2A and 2B. Adding CNS either in combination with pure noise or alone, improves signal quality and mitigates artefact. This improvement in signal quality follows an inverted-U profile, which means that at low added noise amplitudes the signal to noise ratio improves by adding noise, up to a certain amount of added noise. When adding more noise the signal to noise ratio decreases as too much noise will drown the signal.

Those skilled in the art know that in current stimulation paradigms, stimulation signals are typically repetitive square wave pulses. Over time, such a repetitive electrical stimulation signal is dissimilar to the brain's own naturally occurring signals. Such repetitive signals may become less effective as the brain “filters out,” “ignores” or accommodates to the repetitive signal. Hence, a problem with standard electrical stimulation parameters used today is habituation, because the electrical stimulation parameters result in a repetitive electrical signal and thus, the brain habituates to the signal or adapts.

One way to have an electrical stimulation signal to resemble the brain's own signals is to utilize a stimulation paradigm that provides a signal that is correlated to that of the brain's normal signals. It is believed that there exists at least some naturally occurring signals within the nervous system that closely resemble 1/f^{(-β)} (in pathology) and/or 1/f^{β} (in healthy brain). Because of this, the efficacy of CNS signals applied to neuronal tissue is improved as those signals comport more closely to the brain's own signals than current stimulation paradigms in use. The correlation between the CNS signals to the nervous systems own signals mean CNS signals are less likely to lose effectiveness over time.

To further the effectiveness of the given therapy, one can modulate the CNS noise paradigm by adding specific peak frequencies to one or both of the 1/f^{(-β)} and 1/f^{β} components that are known or associated with given brain areas. For example, one can add an alpha frequency peak to one or both CNS′ components to stimulate primary and secondary cortical areas; add a theta frequency peak to one or both CNS′ components to stimulate the cingulate, hippocampus, amygdala; add a delta frequency peak to one or both CNS′ components to stimulate the brainstem, ventral tegmental area (VTA), nucleus accumbens/ventral medial prefrontal cortex (VMPFC).

These additional peak frequencies that are added to the one or both CNS′ components can be obtained from the individual by EEG or MEG measurements or any other measurement to obtain the individual peak frequency or the frequencies can be obtained from a database, for example a database containing a list of given frequencies and spectral structures for a brain structure or brain area. The frequency for each brain area, for example, each Brodmann area, can be easily calculated by defining a Brodmann area in source space and performing a spectral analysis for that area using any software (i.e., sLORETA) to perform source analysis.

Still further, the one or both CNS′ components can be modified by using multiple poles or electrodes. For example, the stimulation paradigm is either sequentially cycled or randomly cycles through the specific or alternating poles or electrodes upon the stimulation lead. CNS can also be selected to specifically activate or inactivate a brain area or brain network. It can be chosen so as to not be normalizing, but instead to be non-physiological to compensate for overactivity or hypoactivity. This can be followed at a later stage with normal physiological CNS parameters.

Subjects to be treated according to some representative embodiments can be selected, identified and/or diagnosed based upon the accumulation of physical, chemical, and historical behavioral data on each patient. One of skill in the art is able to perform the appropriate examinations to accumulate such data. One type of examination can include neurological examinations, which can include mental status evaluations, which can further include a psychiatric assessment. Other types of assessments for movement disorders may include such assessments for example using the Unified Parkinson's Disease Rating Scale (UPDRS). Still further, other types of examinations can include, but are not limited to, motor examination, cranial nerve examination, cognitive assessment and neuropsychological tests (i.e., Minnesota Multiphasic Personality Inventory, Beck Depression Inventory, or Hamilton Rating Scale for Depression). Other types of assessment for tinnitus, for example, can include but are not limited to Numeric Rating Scales (NRS) or Visual Analogue Scales (VAS) and Tinnitus Handicap Inventory (THI), Tinnitus Questionnaire, Tinnitus Functional Index, for pain VAS or NRS, as well as Pain Vigilance and Awareness Questionnaire, Pain Catastrophizing Scale, McGill Pain questionnaire and others, for depression Beck Depression Inventory or Hospital Anxiety and Depression Scale. In addition to neurological testing, routine hematological and/or biochemistry testing may also be performed.

In addition to the above examinations, imaging techniques can be used to determine normal and abnormal brain function that can result in disorders. Thus, once the patient is identified from the above clinical examinations, imaging techniques can be further utilized to provide the region of interest in which the electrodes are to be implanted. Functional brain imaging allows for localization of specific normal and abnormal functioning of the nervous system. This includes electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET) which can be utilized to localize brain function and dysfunction.

### III. Implantation of Stimulation Leads

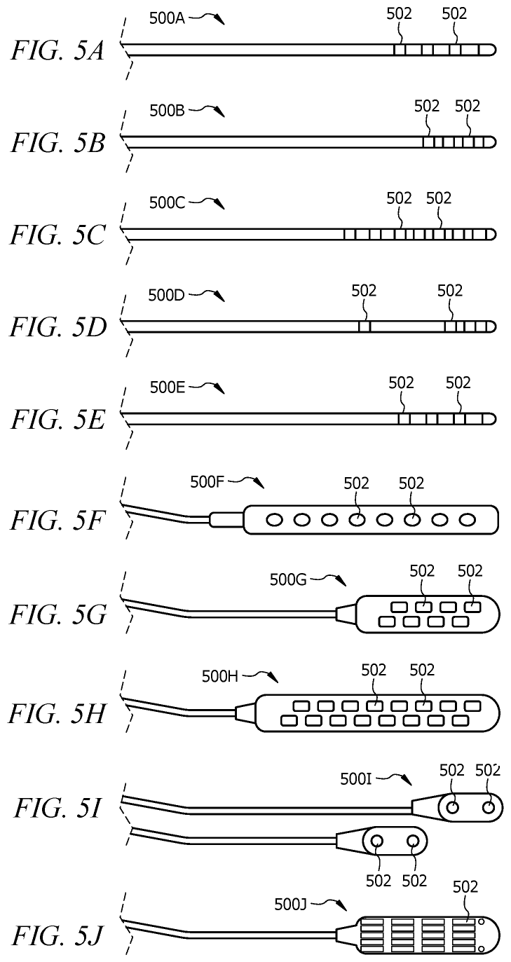

One or more stimulation leads 100, as shown in FIGS. 5A-5J are implanted such that one or more stimulation electrodes 502 of each stimulation lead 500 are positioned or disposed near, adjacent to, directly on or onto, proximate to, directly in or into or within the target tissue or predetermined site. The leads shown in FIG. 5 are exemplary of many commercially available leads, such as deep brain leads, percutaneous leads, paddle leads, etc. Examples of commercially available stimulation leads includes a Nuvectra® percutaneous lead or various laminotomy or paddle leads, such as Nuvectra's laminotomy lead, that are currently on market with which those skilled in the art are familiar. For the purposes described herein and as those skilled in the art will recognize, when an embedded stimulation system, such as the Bion®, is used, it is positioned similar to positioning the lead 500.

Techniques for implanting stimulation electrodes are well known by those of skill in the art and may be positioned in various body tissues and in contact with various tissue layers; for example, deep brain, cortical, subdural, subarachnoid, epidural, cutaneous, transcutaneous and subcutaneous implantation is employed in some embodiments.

### A. Brain

Central neuronal tissue includes brain tissue, spinal tissue or brainstem tissue. Brain tissue can include the frontal lobe, the occipital lobe, the parietal lobe, the temporal lobe, the cerebellum, or the brain stem. More specifically, brain tissue can include subcortical targets, for example, thalamus/sub-thalamus (i.e., thalamic nuclei, medial and lateral geniculate body, intralaminar nuclei, nucleus reticularis, pulvinar, subthalamic nuclei (STN), habenula etc) basal ganglia (i.e., putamen, caudate nucleus, globus pallidus), hippocampus, amygdala, hypothalamus, epithalamus, mammilary bodies, ventral tegmental area (VTA), nucleus accumbens, substantia nigra, corpus callosum, fornix, internal capsula, anterior and posterior commissural, cerebral peduncles etc. Brain tissue also includes cerebellum, cerebellar peduncles, and cerebellar nuclei such as fastigial nucleus, globose nucleus, dentate nucleus, emboliform nucleus. Further, in addition to grey matter, also white matter tracts may be targeted, such as the anterior limb of the interior capsula, the medial forebrain bundle, the cingulum bundle and other white matter tracts connecting different parts of the brain. Still further, in addition to the above-mentioned subcortical targets, brain tissue also includes cortical targets, for example, auditory cortex, prefrontal cortex, the dorsolateral prefrontal cortex, the ventromedial prefrontal cortex, the cingulate cortex, subcallosal area, anterior cingulate cortex, the subgenual anterior cingulate cortex, the motor cortex and the somatosensory cortex. The somatosensory cortex comprises the primary, the secondary somatosensory cortex, and the somatosensory association complex. Still further, the somatosensory cortex also includes Brodmann areas 1, 2, 3, 5, and 7. Yet further, brain tissue can include various Brodmann areas for example, but not limited to Brodmann area 9, Brodmann area 10, Brodmann area 24, Brodmann area 25, Brodmann area 32, Brodmann area 39, Brodmann area 41, Brodmann area 42, and Brodmann area 46.

While not being bound by the description of a particular procedure, patients who are to have an electrical stimulation lead or electrode implanted into the brain for deep brain stimulation, generally, first have a stereotactic head frame, such as the Leksell, CRW, or Compass, mounted to the patient's skull by fixed screws. Subsequent to the mounting of the frame, the patient typically undergoes a series of magnetic resonance imaging sessions, during which a series of two-dimensional slice images of the patient's brain are built up into a quasi-three-dimensional map in virtual space. This map is then correlated to the three-dimensional stereotactic frame of reference in the real surgical field. In order to align these two coordinate frames, both the instruments and the patient must be situated in correspondence to the virtual map. The current way to do this is to rigidly mount the head frame to the surgical table.

Subsequently, a series of reference points are established to relative aspects of the frame and patient's skull, so that either a person or a computer software system can adjust and calculate the correlation between the real world of the patient's head and the virtual space model of the patient MRI scans. The surgeon is able to target any region within the stereotactic space of the brain with precision (e.g., within 0.5 mm). Initial anatomical target localization is achieved either directly using the MRI images or functional imaging (PET or SPECT scan, fMRI, MSI), or indirectly using interactive anatomical atlas programs that map the atlas image onto the stereotactic image of the brain. As is described in greater detail elsewhere in this application, the anatomical targets or predetermined site may be stimulated directly or affected through stimulation in another region of the brain.

In addition to deep brain stimulation, cortical stimulation can also be used to stimulate various brain tissues. Any of the stimulation leads illustrated in FIGS. 5A-5J can be used for cortical stimulation, as well as any other cortical electrode or electrode array. For implanting conventional cortical electrodes, it typically requires a craniotomy under general anesthesia to remove a relatively large (e.g., thumbnail-sized or larger) window in the skull. A pilot hole (e.g., 4 mm or smaller) can be formed through at least part of the thickness of the patient's skull adjacent a selected or predetermined site. In certain embodiments, the pilot hole can be used as a monitoring site.

The location of the pilot hole (and, ultimately the electrode received therein) can be selected in a variety of fashions, for example, the physician may use anatomical landmarks, e.g., cranial landmarks such as the bregma or the sagittal suture, to guide placement and orientation of the pilot hole or the physician may use a surgical navigation system.

Navigation systems may employ real-time imaging and/or proximity detection to guide a physician in placing the pilot hole and in placing the electrode in the pilot hole. In some systems, fiducials are positioned on the patient's scalp or skull prior to imaging and those fiducials are used as reference points in subsequent implantation. In other systems, real-time MRI or the like may be employed instead of or in conjunction with such fiducials. A number of suitable navigation systems are commercially available, such as the STEALTHSTATION TREON TGS sold by Medtronic Surgical Navigation Technologies of Louisville, Colo., U.S.

Once the pilot hole is formed, the threaded stimulation lead may be advanced along the pilot hole until the contact surface electrically contacts a desired portion of the patient's brain. If the stimulation lead is intended to be positioned epidurally, this may comprise relatively atraumatically contacting the dura mater; if the electrode is to contact a site on the cerebral cortex, the electrode will be advanced to extend through the dura mater. Thus, the lead may be placed epidurally or subdurally for cortical stimulation.

B. Spinal Cord and/or Peripheral Nerves

Peripheral nerves can include, but are not limited to cranial nerves, somatic (spinal) nerves and autonomic nerves. Cranial nerves include the olfactory nerve, optic nerve, oculomotor nerve, trochlear nerve, trigeminal nerve, abducens nerve, facial nerve, vestibulocochlear (auditory) nerve, glossopharyngeal nerve, vagal nerve, accessory nerve, and hypoglossal nerve. Peripheral spinal nerves can be any nerve from C2 to S5, i.e. cervical, thoracic, lumbar and sacral nerves, and include both afferent and efferent components. Some spinal nerve branches have names such as occipital nerve (e.g., suboccipital nerve, the greater occipital nerve, the lesser occipital nerve), the greater auricular nerve, the lesser auricular nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, genitofemoral nerves, and iliohypogastric nerves. Furthermore, peripheral neuronal tissue can include but is not limited to peripheral nervous tissue associated with a dermatome. In other embodiments the peripheral nerve can be an autonomic nerve, either from the sympathetic or parasympathetic system. Some of the autonomic nerves run in named nerves such as the vagus (=vagal) nerve, the different splanchnic nerves, celiac nerve, mesenteric plexus, phrenic nerve. In other embodiments the stimulation can be applied to the dorsal root ganglion of any one or more cervical (C1, C2, C3, C4, CS, C6,C7 and C8), thoracic (T1, T2, T3, T4, T5, T6, T7, T8, T9, T10, T11, T12), lumbar (L1, L2, L3,L4. L5) or sacral (S1, S2, S3, S4, S5) nerves.

Spinal tissue can include the ascending and descending tracts of the spinal cord, more specifically, the ascending tracts that comprise intralaminar neurons or the dorsal column. For example, the spinal tissue can include neuronal tissue associated with any of the cervical vertebral segments (C1, C2, C3, C4, CS, C6, C7 and C8) and/or any tissue associated with any of the thoracic vertebral segments (T1, T2, T3, T4, T5, T6, T7, T8, T9, T10, T11, T12) and/or any tissue associated with any of the lumbar vertebral segments (L1, L2, L3, L4. L5) and/or any tissue associated with the sacral vertebral segments (S1, S2, S3, S4, S5). More specifically, the spinal tissue is the dorsal column or the dorsal root ganglia of the spinal cord. The brainstem tissue can include the medulla oblongata, pons or mesencephalon, more particular the posterior pons or posterior mesencephalon, Lushka's foramen, and ventrolateral part of the medulla oblongata.

In other embodiments, the stimulation leads are positioned in communication with the neuronal tissue of the spinal cord, more specifically, the dorsal root ganglia or the dorsal column of the spinal cord. For example, stimulation electrodes are commonly positioned external to the dura layer surrounding the spinal cord. Stimulation on the surface of the cord is also contemplated, for example, stimulation may be applied to the spinal cord tissue as well as to the nerve root entry zone. Stimulation electrodes may be positioned in various body tissues and in contact with various tissue layers; for example, subdural, subarachnoid, epidural, and cutaneous, and/or subcutaneous implantation is employed in some embodiments.

Spinal cord stimulation can be accomplished utilizing either percutaneous leads and/or laminotomy type leads that comprise a paddle. Percutaneous leads commonly have two or more equally spaced electrodes which are placed above the dura layer through the use of a Touhy-like needle. For insertion, the Touhy-like needle is passed through the skin between desired vertebrae to open above the dura layer.

In contrast to the percutaneous leads, laminotomy leads have a paddle configuration and typically possess a plurality of electrodes (for example, two, four, eight, sixteen or thirty-two) arranged in one or more columns. Implanted laminotomy leads are commonly transversely centered over the physiological midline of a patient. In such position, multiple columns of electrodes are well suited to address both unilateral and bilateral pain, where electrical energy may be administered using either column independently (on either side of the midline) or administered using both columns to create an electric field which traverses the midline. A multi-column laminotomy lead enables reliable positioning of a plurality of electrodes, and in particular, a plurality of electrode columns that do not readily deviate from an initial implantation position.

Laminotomy leads require a surgical procedure for implantation. The surgical procedure, or partial laminectomy, requires the resection and removal of certain vertebral tissue to allow both access to the dura and proper positioning of a laminotomy lead. The laminotomy lead offers a more stable platform, which is further capable of being sutured in place that tends to migrate less in the operating environment of the human body. Depending on the position of insertion, however, access to the dura may only require a partial removal of the ligamentum flavum at the insertion site. In some embodiments, two or more laminotomy leads may be positioned within the epidural space, and the leads may assume any relative position to one another.

In certain embodiments, the stimulation leads may be placed subcutaneously on the patient's head. For example, one or more stimulation leads can be implanted subcutaneously such that one or more stimulation electrodes are positioned in communication with a dermatome area, for example (C1, C2, C3) or cervical nerve roots (e.g., C1, C2, C3) or cranial nerves (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and hypoglossal nerve) and/or occipital area For example, one or more stimulation electrodes are positioned in the C2 dermatome area/C3 dermatome area, subcutaneously, but superior to the galea. Within certain areas of the C2 dermatome area or occipital or occiput area, there is little or no muscle, this area primarily consists of fat, fascia, perostium, and neurovascular structures (e.g., galea). More specifically, the electrode can be implanted in a subcutaneous fashion such that the electrode is positioned below the skin, above the bone on the back of the head or superior to the periosteum. On the back of the head, the probe is positioned in the C2 dermatome area or positioned at the back of the patient's head at about the level of the ear.

### C. Brainstem Stimulation

Implantation of a stimulation lead in communication with the predetermined brainstem area can be accomplished via a variety of surgical techniques that are well known to those of skill in the art. For example, an electrical stimulation lead can be implanted on, in, or near the brainstem by accessing the brain tissue through a percutaneous route, an open craniotomy, or a burr hole. Where a burr hole is the means of accessing the brainstem, for example, stereotactic equipment suitable to aid in placement of an electrical stimulation lead on, in, or near the brainstem may be positioned around the head. Another alternative technique can include, a modified midline or retrosigmoid posterior fossa technique.

In certain embodiments, electrical stimulation lead is located at least partially within or below the dura mater adjacent the brainstem. Alternatively, a stimulation lead can be placed in communication with the predetermined brainstem area by threading the stimulation lead up the spinal cord column, as described above, which is incorporated herein.

Still further, a predetermined brainstem area can be indirectly stimulated by implanting a stimulation lead in communication with a cranial nerve (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and the hypoglossal nerve) as well as high cervical nerves (cervical nerves have anastomoses with lower cranial nerves) such that stimulation of a cranial nerve indirectly stimulates the predetermined brainstem tissue. Such techniques are further described in U.S. Pat. Nos. 6,721,603; 6,622,047; and 5,335,657 each of which are incorporated herein by reference.

### IV. Generation of Stimulation Parameters and Modifications Thereof

Conventional IMDs can be modified to apply CNS, or CNS in combination with individual peak frequencies (e.g., alpha, theta and delta) or combination of CNS combined with burst or tonic stimulation to nerve tissue of a patient by modifying the software instructions and/or stimulation parameters stored in the devices. Specifically, conventional IMDs typically include a microprocessor and a pulse generation module. The pulse generation module generates the electrical pulses according to a defined pulse width and pulse amplitude and applies the electrical pulses to defined electrodes through switching circuitry and the wires of a stimulation lead. The microprocessor controls the operations of the pulse generation module according to software instructions stored in the device and accompanying stimulation parameters. Examples of commercially available IMDs that can be modified according to some embodiments include the Precision® marketed and sold by Boston Scientific. Other IMDs can include, Algovita® formerly marketed and sold by Nuvectra, Nalu® Neurostimulation System, or Medtronic's Activa® marketed and sold by Medtronic.

These IMDs can be adapted by modifying the software instructions provided within the IMDs used to control the operations of the devices. In some embodiments, software is provided within the IMD to retrieve or generate a stream of digital values that define a waveform according to the desired power spectral density. This stream of values is then employed to control the amplitude of successive stimulation pulses generated by the IMD. The software may include a pseudo-random number generator according to known algorithms to generate the stream of digital values. Alternatively, one or more streams of digital values having the desired power spectral density may be generated offline and stored in memory of the IMD (in a compressed or other suitable format). The software of the IMD may retrieve the values from memory for control of the amplitude of the output pulses of the IMD. Alternatively, an external conventional IMD can be used (for example, the DS8000™ digital stimulator available from World Precision Instruments) to generate the desired electrical stimulation. For example, a custom waveform may be generated offline on a personal computer and imported into the digital stimulator for pulse generation. Signal parameters may be inputted, such as CNS spectrum, for example FIG. 4 at 407 or 425, into suitable waveform generating software to generate the stream of digital values. Alternatively, depending upon the capabilities of the external digital stimulator, the stream of digital values may be calculated on board the processor of the external digital stimulator.

FIG. 7 depicts an exemplary IMD that can be used to provide the desired stimulation. Signal parameters are inputted, such as CNS spectrum, as shown for example in. FIG. 4 at 417, 419, 421, 423, and 425, or FIG. 4 at 407, 40, 411, 413 and 415 into the software or memory 710 and the desired wave pattern or signals are generated using microprocessor 720. A standard digital-to-analog converter 730 receives the calculated digital signals and generates analog output pulses corresponding to the values of the digital signals. The generated output pulses may be outputted from the IMD through an output capacitor. Optionally, any suitable filter 740 can be used to smooth or shape the signals; however, unsmoothed or unfiltered signals can be transmitted to the switching circuitry 750 which provides the signals to the electrodes 500 thereby stimulating the neuronal tissue using the desired CNS pattern.

Another example of the IMD stimulator design is disclosed in U.S. Pat. No. 8,996,117, the entire disclosure of which is incorporated herein by reference, which discloses a waveform generator with scalable waveform features. The IMD disclosed in U.S. Pat. No. 8,996,117 the entire disclosure of which is incorporated herein by reference, is well suited to deliver the CNS waveforms. Those skilled in the art recognize that other IMDs including those mentioned herein and those on the market that are not specifically mentioned are able to deliver the CNS waveform as well. For example purposes only, rather than restate U.S. Pat. No. 8,996,117 herein, the patent is incorporated by reference herein in full. As those skilled in the art will appreciate, the example IMD in U.S. Pat. No. 8,996,117 would be able to provide a representation of CNS as shown in FIG. 4 at 403. FIG. 4 at 403 illustrates an exemplary waveform generated by the waveform generators disclosed therein, and that provide the electrodes with CNS to stimulate neuronal tissue.

In addition to providing a stimulation waveform similar to that of CNS spectrum; it may be desirable to modify the CNS waveform stimulation pattern. Such modifications can utilize the addition of peak frequencies, such as the addition of an alpha, beta, theta, and/or delta peaks to CNS spectrum waveform. Such frequency peaks can be obtained by using standard peaks or individualizing the frequency peaks. Such information can be communicated to the microprocessor 720 via the software component 710. Thus, the data communicated can comprise standard frequency peaks or comprise individualized frequency peaks or patient specific. The patient specific frequency peaks can be obtained off-line or in real time or on-line, for example prior to implantation or at any time point after implantation, for example, during the initial programming of the IMD. Any suitable signal processing technique may be employed to add the appropriate spectral peaks. For example, a suitable filter may be applied to the noise signal. Alternatively, a separate signal may be generated with a spectral peak about the desired frequency and the separate signal may be added to or superimposed on the noise signal.

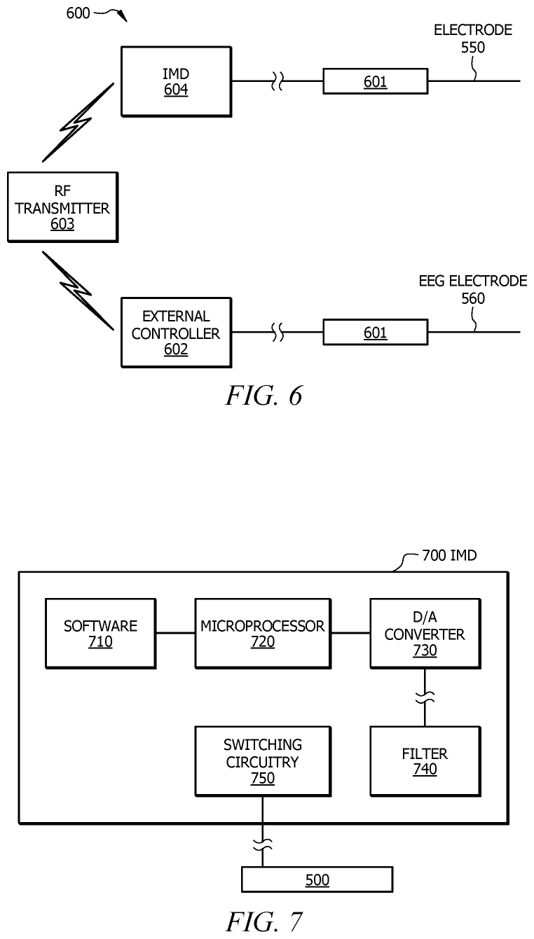

With reference to FIG. 6, with electrodes disposed near, adjacent to directly next to or within the target neuronal tissue, for example, brain tissue, some representative embodiments utilize the detection and analysis of neuronal activity, such as EEG measurements. Specifically, terminals of the lead, such as an EEG lead, may be coupled to electrodes 560 using respective conductors 601 to external controller that contains suitable circuitry to analyze neuronal activity, for example, an EEG analyzer can be included in the external controller in which the analyzer functions are adapted to receive EEG signals from the electrodes and process the EEG signals to identify frequency peaks, such as LORETA software can be used. Further signal processing may occur on a suitable computer platform within the external controller using available signal processing. The computer platform may include suitable signal processing algorithms (e.g., time domain segmentation, FFT processing, windowing, logarithmic transforms, etc.). Further platforms or algorithms to modify the signals are included in the modification algorithms (e.g., envelope modification, etc). User interface software may be used to present the processed neuronal activity (i.e., specific peak frequency) and combine a specific peak frequency with the CNS waveform patterns to the transmitter 603 which then transmits, for example, via radio frequency to the IMD 604 which is adapted to provide the CNS waveform patterns with the peak frequency to achieve stimulation of the target neuronal tissue via electrode 550. This procedure can be performed on-line or off-line. Additionally, IMD 604 preferably comprises circuitry such as an analog-to-digital (AD) converter, switching circuitry, amplification circuitry, transmitters, and/or filtering circuitry.

Still further, it may be desirable to utilize an implantable device that is capable of performing only the functions of the external controller or that is capable of performing the functions of the external controller and the functions of an IMD, all in one. When the implantable device is only performing only those functions of the external controller, those of skill in the art can modify an implantable device such that it is capable of detecting/sampling and processing of the signal's representative of the neuronal activity/EEG activity. Such a device may include a microprocessor that is capable of performing these activities as well as a transmitter such that the signals can be transmitted via radiofrequency to another implantable device, such as described above in FIG. 6 that is capable of generating the desired signal to the target tissue. Thus, an EEG lead is placed or positioned near the target brain tissue via methods known to those of skill in the art. The EEG lead detects neuronal activity which is relayed to the processor that possesses sufficient computational capacity to collect the information obtained from the EEG electrode, process it to obtain the respective frequency peak desired and/or modulate the frequency peaks and transmit the frequency to an RF transmitter that transmits the respective information to microprocessor located in the stimulation IMD. Those skilled in the art will realize that for the combined IMD, the information will be processed by a microcontroller within the IMD and transferred to the correct processing circuitry to obtain the respective frequency peak desired and/or modulate the frequency peaks and stimulate according to the invention as described herein.

Another means to modify the CNS waveform pattern is to combine it with any variety of waveforms, such as sinewaves, arbitrary waveforms, tonic waveforms, or burst waveforms. Thus, an IMD can be implemented to apply either a variety of waveforms using a digital signal processor and one or several digital-to-analog converters. The waveforms could be defined in memory and applied to the digital-to-analog converter(s) for application through electrodes of the medical lead. The digital signal processor could scale the various portions of the waveform in amplitude and within the time domain (e.g., for the various intervals) according to the various waveform parameters. A doctor, the patient, or another user of waveform source may directly or indirectly input waveform parameters to specify or modify the nature of the waveforms provided. An example system able to perform this function is the former Nuvectra's Algovita system.

Thus, a microprocessor and suitable software instructions to implement the appropriate system control can be used to control waveform therapy in combination with CNS. The processor can be programmed to use “multiple programs sets” which are known in the art. A “program set” refers to a set of waveform parameters which define a waveform's pulse to be generated. For example, a program set defines the waveform's pulse amplitude, a pulse width, a pulse delay, pulse shape, frequency, and an electrode combination among other such parameters. The pulse amplitude refers to the amplitude for a given pulse and the pulse width refers to the duration of the pulse. The pulse delay represents an amount of delay to occur after the generation of the pulse (equivalently, an amount of delay could be defined to occur before the generation of a pulse). The waveform shape represents the design or shape of the waveform. The amount of delay represents an amount of time when no pulse generation occurs. The electrode combination defines the polarities for each output which, thereby, controls how a pulse is applied via electrodes of a lead. Other pulse parameters could be defined for each program set such as pulse type, repetition parameters, etc. Still further, the CNS waveform pattern alone or in combination with other waveforms may be implemented such that the stimulation occurs either sequentially, randomly or pseudo-sequentially over multiple poles or electrodes on the lead.

In certain embodiments, the waveform parameters may comprise atype of stimulation having a frequency in the range of about 0.01 Hz to about 1000 Hz in combination with a different type of stimulation having a frequency in the range of about 0.01 Hz to about 1000 Hz. Those of skill in the art realize that the frequencies can be altered depending upon the capabilities of the IMDs that are utilized. More particularly, for example, burst stimulation may be at about 0.01 to 100 Hz consisting of 2 to 10 spikes with 1 ms to 10 ms pulse width, 0.1 to 100 ms interspike interval in combination with CNS signals interspersed between or around the burst or prior to or after the burst or in any variation thereof depending upon the efficacy of treatment. Still further, CNS signals or stimulation paradigm as described herein may be used in combination with about 0.01 to 1000 Hz tonic stimulation interspersed between or around the CNS signals or stimulation paradigm, or any variation thereof depending upon the efficacy of treatment and the capabilities of the IMD.

Still further, those of skill in the art recognize that burst firing refers to an action potential that is a burst of high frequency spikes (50-1000 Hz) (Beurrier et al., 1999). Burst firing acts in a non-linear fashion with a summation effect of each spike and tonic firing refers to an action potential that occurs in a linear fashion.

Yet further, a particular stimulation paradigm can refer to a period in the stimulation train that has a much higher discharge rate than surrounding periods in the spike train (N. Urbain et al., 2002). An example of a particular paradigm, burst can refer to a plurality of groups of spike pulses. A burst is a train of action potentials that, possibly, occurs during a ‘plateau’ or ‘active phase’, followed by a period of relative quiescence called the ‘silent phase’ (Nunemaker, Cellscience Reviews Vol 2 No. 1, 2005.) Thus, a burst paradigm may comprise of spikes having an inter-spike interval in which the spikes are separated by 0.1 milliseconds to about 100 milliseconds. Those of skill in the art realize that the inter-spike interval can be longer or shorter. Yet further, those of skill in the art also realize that the spike rate within the burst does not necessarily occur at a fixed rate; this rate can be variable. A spike refers to an action potential. Yet further, a “burst spike” refers to a spike that is preceded or followed by another spike within a short time interval (Matveev, 2000), in other words, there is an inter-spike interval, in which this interval is generally about 100 ms but can be shorter or longer, for example 0.1 milliseconds.

Still further, it may be of interest to use a system that includes a processor that determines whether the patient is in a sleep state, and controls therapy based upon the sleep state. The sleep state may be relevant for the CNS therapy if during a given sleep stage the patient's frequency spectrum changes, for example, the high frequency component in gamma is decreased. In this embodiment IMD may automatically adjust the value of β for one or both components of the CNS from a value of 1 to a value of 2 or even steeper stimulation with β>−2.

As referred to herein, the sleep state may refer to a state in which patient is intending on sleeping (e.g., initiating thoughts of sleep), is at rest, is attempting to sleep or has initiated sleep and is currently sleeping. In addition, the processor may determine a sleep stage of the sleep state based on a biosignal detected within brain the patient and control therapy delivery to patient based on a determined sleep stage. Examples of biosignals include, but are not limited to, electrical signals generated from local field potentials within one or more regions of brain, such as, but not limited to, an electroencephalogram (EEG) signal or an electrocorticogram (ECOG) signal. The biosignals that are detected may be detected within the same tissue site of brain as the target tissue site for delivery of electrical stimulation. In other examples, the biosignals may be detected within another tissue site.

Within a sleep state, the patient may be within one of a plurality of sleep stages. Example sleep stages include, for example, Stage 1 (also referred to as Stage N1 or S1), Stage 2 (also referred to as Stage N2 or S2), Deep Sleep (also referred to as slow wave sleep), and rapid eye movement (REM). The Deep Sleep stage may include multiple sleep stages, such as Stage N3 (also referred to as Stage S3) and Stage N4 (also referred to as Stage S4). In some cases, the patient may cycle through the Stage 1, Stage 2, Deep Sleep, REM sleep stages more than once during a sleep state. The Stage 1, Stage 2, and Deep Sleep stages may be considered non-REM (NREM) sleep stages.

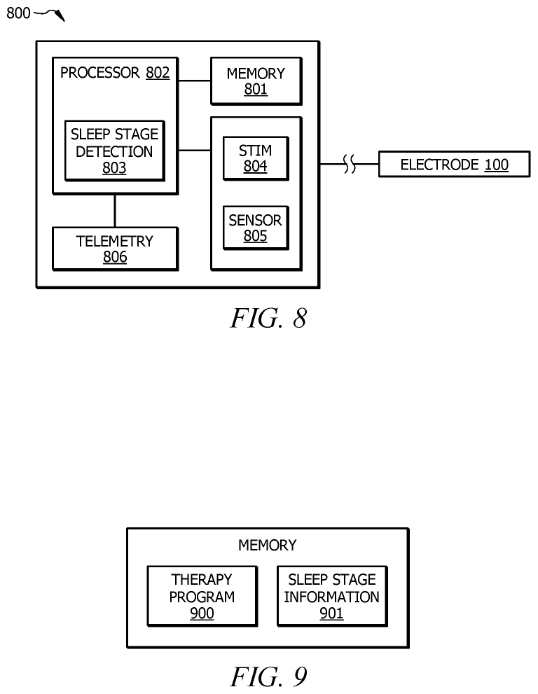

FIG. 8 shows an exemplary implantable IMD 800 that can be used to determine a stage of sleep and adjust therapy. For example, the device may include, processor 802, memory 801, stimulation generator 804, sensing module 805, telemetry module 806, and sleep stage detection module 803. Although sleep stage detection module 803 is shown to be a part of processor 802 in FIG. 7, in other examples, sleep stage detection module 803 and processor 802 may be separate components and may be electrically coupled, e.g., via a wired or wireless connection.

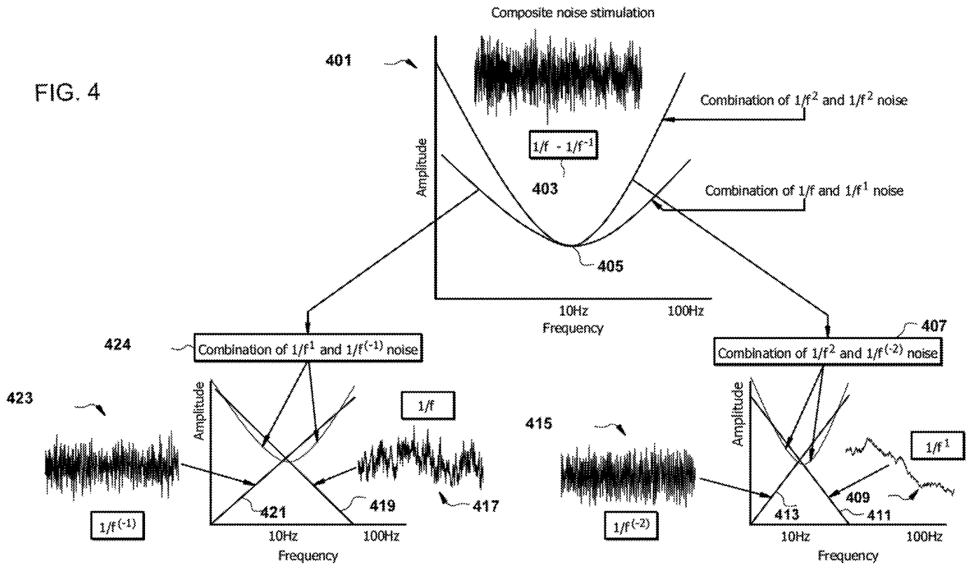

Memory 901, as shown in FIG. 9, may include any volatile or non-volatile media, such as a random access memory (RAM), read only memory (ROM), non-volatile RAM (NVRAM), electrically erasable programmable ROM (EEPROM), flash memory, and the like.

Memory 801 may store instructions for execution by processor 802 and information defining therapy delivery for the patient, such as, but not limited to, therapy programs or therapy program groups, information associating therapy programs with one or more sleep stages, thresholds or other information used to detect sleep stages based on biosignals, and any other information regarding therapy of the patient. Therapy information may be recorded in memory 801 for long-term storage and retrieval by a user. As described in further detail with reference to FIG. 9, memory 801 may include separate memories for storing information, such as separate memories for therapy programs 900, and sleep stage information 901. Yet further, other memories that may be stored may include patient information, such as information relating to specific peak frequencies, or information relating to CNS.

It is also envisaged that the recording electrode can be used to record or detect sleep stage or when a subject is not in a sleep stage, the recording electrode can be used to detect a change in the normal spectral composition of the noise and adjust the parameters of the stimulation therapy, for example, adjust the stimulation factors such as drowsiness, stress, depression, excitement, arousal, alcohol or other drug intake etc.

### V. Treating Neurological Conditions

The stimulation method of the present invention acts to modulate neuronal tissue which in turn modulates the neuronal tissue to cause/allow the tissue to act in the best interest of the host through use of the its natural mechanisms.

Accordingly, the present methods and/or devices relate to modulation of neuronal activity to affect neurological, neuropsychological or neuropsychiatric activity. The present method finds particular application in the modulation of neuronal function or processing to affect a functional outcome. The modulation of neuronal function is particularly useful with regard to the prevention, treatment, or amelioration of neurological, psychiatric, psychological, conscious state, behavioral, mood, and thought activity or connectivity (unless otherwise indicated these will be collectively referred to herein as “neurological activity” which includes “psychological activity” or “psychiatric activity”). When referring to a pathological or undesirable condition associated with the activity or connectivity, reference may be made to a neurological disorder which includes “psychiatric disorder” or “psychological disorder” instead of neurological activity or connectivity or psychiatric or psychological activity or connectivity. Although the activity and/or connectivity to be modulated usually manifests itself in the form of a disorder such as an attention or cognitive disorders (e.g., Autistic Spectrum Disorders); mood disorder (e.g., major depressive disorder, bipolar disorder, and dysthymic disorder) or an anxiety disorder (e.g., panic disorder, posttraumatic stress disorder, obsessive-compulsive disorder and phobic disorder); neurodegenerative diseases (e.g., multiple sclerosis, Alzheimer's disease, amyotrophic lateral sclerosis (ALS), Parkinson's disease, Huntington's disease, Guillain-Barre syndrome, myasthenia gravis, and chronic idiopathic demyelinating disease (CID)), movement disorders (e.g., dyskinesia, tremor, dystonia, chorea and ballism, tic syndromes, Tourette's syndrome, myoclonus, drug-induced movement disorders, Wilson's disease, Paroxysmal Dyskinesias, Stiff Man Syndrome and Akinetic-Ridgid Syndromes and Parkinsonism), epilepsy, tinnitus, pain, phantom pain, diabetes neuropathy, addiction (both substance and non-substance related), one skilled in the art appreciates that the invention may also find application in conjunction with enhancing or diminishing any neurological or psychiatric function, not just an abnormality or disorder. Neurological activity that may be modulated can include, but not be limited to, normal functions such as alertness, conscious state, drive, fear, anger, aggression, anxiety, repetitive behavior, impulses, urges, obsessions, euphoria, sadness, and the fight or flight response, as well as instability, vertigo, dizziness, fatigue, photophobia, concentration dysfunction, memory disorders, including dementias such as Alzheimer or Lewy body disease, headaches and facial pains, dizziness, irritability, fatigue, visual disturbances, sensitivity to noise (misophonia, hyperacusis, photophobia), judgment problems, depression, symptoms of traumatic brain injury (whether physical, emotional, social or chemical), autonomic functions, which includes sympathetic and/or parasympathetic functions (e.g., control of heart function, intestinal function), somatic functions, and/or enteric functions. Thus, the present methods and/or devices encompass modulation of central and/or peripheral spinal and autonomic nervous systems. In another embodiment the stimulation may be used to treat personality disorders, including Cluster A: paranoid, Schizoid and Schizotypal, Cluster B (dramatic): Antisocial, Borderline, Histrionic, Narcissistic, and Cluster C: (anxious) Avoidant, Dependent, Obsessive compulsive or Not specified: Depressive, Haltlose, Passive-aggressive, Sadistic, Self-defeating and Psychopathic personality disorders. In this embodiment the stimulation may be associated with psychedelic drugs to obtain a better result.

Other neurological disorders can include, but are not limited to headaches, for example, migraine, trigeminal autonomic cephalgia (cluster headache (episodic and chronic)), paroxysmal hemicrania (episodic and chronic), hemicrania continua, SUNCT (short-lasting unilateral neuralgiform headache with conjunctival injection and tearing), cluster tic syndrome, trigeminal neuralgia, tension type headache, idiopathic stabbing headache, etc. The neurostimulation device can be implanted intracranially or peripherally, for example, but not limited to implanting a neurostimulation device occipitally or frontally for the treatment of headaches and facial pain.

Autonomic and/or enteric nervous system disorders that can be treated using the stimulation system and/or method of the present invention include, but are not limited to hypertension, neurosis cordis or heart rhythm disorders, obesity, gastrointestinal motion disorders, respiratory disorders, diabetes, sleep disorders, snoring, incontinence both urologic and gastrointestinal, sexual dysfunction, chronic fatigue syndrome, fibromyalgia, whiplash associated symptoms, post-concussion syndrome, posttraumatic stress disorder etc.

Yet further, immunological disorders may also be treated using the stimulation system and/or method of the present invention. This is based on the fact that the immune system senses antigens coordinates' metabolic, endocrine and behavioral changes that support the immune system and modulates the immune system via neuroendocrine regulation and direct immune cell regulation. Such immunological disorders include, such as allergy, rhinitis, asthma, rheumatoid arthritis, psoriasis arthritis, lupus erythematosus disseminatus, multiple sclerosis and other demyelinating disorders, autoimmune thyroiditis, Crohn's disease, diabetes mellitus etc. in this embodiment the stimulation may be at the autonomic nerves, the vagus nerve, C2 or C2 nerve, the spinal cord or inside the brain where the central control is regulated, for example the posterior cingulate cortex, anterior cingulate cortex, insula, hypothalamus.

Still further tumoral disorders, both malignant and benign may also be treated using the stimulation system and/or method of the present invention. This is based on the fact that tumoral behavior is linked to immunological function. This is seen in immunodeficiency syndromes such as AIDS and hematological disorders, where multiple and different tumors develop. In this setting neuromodulation could indirectly influence tumoral behavior.

Yet further neuroendocrine disorders may also be treated using the stimulation system and/or method of the present invention. Such disorders are stress reactions, hypothalamic-pituitary axis dysfunction, etc.

Yet further functional disorders may also be treated using the stimulation system and/or method of the present invention. Such disorders can be anorexia, bulimia, phobias, addictions, paraphilia, psychosis, depression, bipolar disorder, kleptomania, aggression, or antisocial sexual behavior. One skilled in the art appreciates that the invention may also find application in conjunction with enhancing or diminishing any neurological or psychiatric function, not just an abnormality or disorder.

Using the above described stimulation system, the predetermined site or target area is stimulated in an effective amount or effective treatment regimen to decrease, reduce, modulate or abrogate the neurological disorder or condition. Thus, a subject or patient is administered a therapeutically effective stimulation so that the subject has an improvement in the parameters relating to the neurological disorder or condition including subjective measures such as, for example, neurological examinations and neuropsychological tests (e.g., Minnesota Multiphasic Personality Inventory, Beck Depression Inventory, Mini-Mental Status Examination (MMSE), Hamilton Rating Scale for Depression, Wisconsin Card Sorting Test (WCST), Tower of London, Stroop task, MADRAS, CGI, N-BAC, or Yale-CNS Obsessive Compulsive score (Y-BOCS)), motor examination, visual analog scale (VAS) and cranial nerve examination, and objective measures including use of additional psychiatric medications, such as anti-depressants, or other alterations in cerebral blood flow or metabolism and/or neurochemistry.

Patient outcomes may also be tested by health-related quality of life (HRQL) measures: Patient outcome measures that extend beyond traditional measures of mortality and morbidity, to include such dimensions as physiology, function, social activity, cognition, emotion, sleep and rest, energy and vitality, health perception, normal eating habits or behaviors (i.e., regained appetite or reduced appetite) and general life satisfaction. (Some of these are also known as health status, functional status, or quality of life measures).

Treatment regimens may vary as well, and often depend on the health and age of the patient. Obviously, certain types of disease will require more aggressive treatment, while at the same time; certain patients cannot tolerate more taxing regimens. The clinician will be best suited to make such decisions based on the known subject's history.

For purposes of this invention, beneficial or desired clinical results include, but are not limited to, alleviation of symptoms, improvement of symptoms, diminishment of extent of disease, stabilized (i.e., not worsening) state of disease, delay or slowing of disease progression, amelioration or palliation of the disease state, and remission (whether partial or total), whether objective or subjective. The improvement is any observable or measurable improvement, or any subjective improvement reported by the implanted person. Thus, one of skill in the art realizes that a treatment may improve the patient condition but may not be a complete cure of the disease.

In certain embodiments, in connection with improvement in one or more of the above or other neurological disorders, the electrical stimulation may have a “brightening” effect on the person such that the person looks better, feels better, moves better, thinks better, and otherwise experiences an overall improvement in quality of life, (e.g., self-confidence, alleviating shyness, distrust etc). In another embodiment this stimulation may be used as enhancement of normal functions if the person desires so.

In certain embodiments, the neuromodulation method described herein is utilized to treat a subject suffering from or suspected of suffering from tinnitus. Thus, a subject is administered a therapeutically effective stimulation so that the subject has an improvement in the parameters relating to tinnitus including informal questioning of the subject, formal subjective testing and analysis according to one or more audiology test, for example the Goebel tinnitus questionnaire or other validated tinnitus questionnaires, audiometry, tinnitus matching, impedance, BAEP, and OAE. The improvement is any observable or measurable improvement. Thus, one of skill in the art realizes that a treatment may improve the patient condition but may not be a complete cure of the disease.

In other embodiments, the neuromodulation method described herein is utilized to treat a subject suffering from or suspected of suffering from pain (chronic or otherwise). One example of a method for pain measurement is the use of the Visual Analog Scale (VAS). In the VAS patients are asked to rank their pain by making a mark on a bar that is labeled “no pain” on one end, and “pain as bad as possible” on the other end. Patients may mark the bar anywhere between the two opposite poles of perceived pain sensation. This mark can then be given any quantitative value such as fractional, decimal or integer values by the clinician and used as a semi-quantitative pain measurement. In various tests for pain severity, patients may rank their pain on a scale between zero and ten, by a scale of faces depicting various emotions from happy to very sad and upset, and by answering a variety of questions describing the pain. In preferred embodiments, the patient's pain is assessed prior to and during a trial implantation procedure. In other embodiments, informal subjective questioning of the person, and/or formal subjective testing and analysis may be performed to determine whether the subject's pain has sufficiently improved throughout trial stimulation.

In addition to utilizing pain scores and grading and objective measures including use of additional pain medications (e.g., reduction in the amount of medication consume or elimination of the consumption of pain medications), other methods to determine improvement of a patient's pain may comprise administering various standardized questionnaires or tests to determine the patient's neuropsychological state as described above.

If the subject's neurological disorder/disease has not sufficiently improved, or if the reduction of the neurological disorder/disease is determined to be incomplete or inadequate during an intra-implantation trial stimulation procedure, stimulation lead may be moved incrementally or even re-implanted, one or more stimulation parameters may be adjusted, or both of these modifications may be made and repeated until at least one symptom associated with the neurological disorder/disease has improved.

Where appropriate, post-implantation trial stimulation may be conducted to determine the efficacy of various types of burst and tonic stimulation. Examples of efficacy metrics may include the minimum required voltage for a given protocol to achieve maximum and/or therapeutic benefits to the neurological disease and/or disorder. Efficacy metrics may also include a measurement of the presence and/or degree of habituation to a given protocol over one or more weeks or months, and any necessary modifications made accordingly. Such assessments can be conducted by any suitable programming device. One such as suitable device is that described in U.S. Pat. No. 9,144,680, which is incorporated by reference here in full. Utilizing such a program allows an optimal stimulation therapy to be obtained at minimal power. This ensures a longer battery life for the implanted systems.

In certain embodiments, it may be desirable for the patient to control the therapy to optimize the operating parameters to achieve increased or optimized the treatment. For example, the patient can alter the pulse frequency, pulse amplitude and pulse width using a handheld radio frequency or Bluetooth or wifi enabled device that communicates with the IMD. Once the operating parameters have been altered by the patient, the parameters can be stored in a memory device to be retrieved by either the patient or the clinician. Yet further, particular parameter settings and changes therein may be correlated with particular times and days to form a patient therapy profile that can be stored in a memory device. In another embodiment remote programming via the cloud may be performed by the treating physician or health care provider.

As will be recognized by those skilled in the art, the innovative concepts described in the present application can be modified and varied over a wide range of applications due to the various parameters that have been described herein, which by selecting or varying one or another, will result in a great number of parameter permutations, thereby presenting a significant number of differing treatment approaches applicable to the needs of individual patients. Accordingly, the scope of patented subject matter should not be limited to any of the specific exemplary teachings discussed above but is instead defined by the following claims.

What is claimed is:

1. A method of stimulating nerve tissue of a patient in need thereof by using an implantable medical device, said method comprising the steps of:
(a) generating a stimulus comprising a noisy signal, wherein said noisy signal comprises a combination of one or more frequency spectrums having a power spectral density per unit of bandwidth proportional to 1/f^{(-β)} noise and of one or more frequency spectrums having a power spectral density per unit of bandwidth proportional to 1/f^{(β)} noise, wherein f is signal frequency and wherein β is any real number but that excludes 0, wherein said noisy signal is generated by a pulse generator of said implantable medical device;
(b) delivering said stimulus to at least one stimulation lead; and
(c) applying said stimulus to nerve tissue of said patient through one or more electrodes of at least one stimulation lead.

2. The method of claim 1, wherein β=1.

3. The method of claim 1, wherein β=2.

4. The method of claim 1, wherein β>2.

5. The method of claim 1, wherein said stimulus is combined with at least one pulse stimulus to be repeated in a tonic manner.

6. The method of claim 1, wherein said stimulus is combined with a burst stimulus that comprises a plurality of groups of spike pulses each individually charge balanced or charge balanced at the end of the group of monophasic spikes.

7. The method of claim 1, wherein said stimulus is combined with at least one pulse stimulus to be repeated in a tonic manner and with a burst stimulation that comprises a plurality of groups of spike pulses each individually charge balanced or charge balanced at the end of the group of monophasic spikes.

8. The method of claim 1, wherein said stimulus is modulated at a selected signal frequency by a step selected from the group comprising of:
(a) selective power increase;
(b) envelope modulation;
(c) addition of more tonic stimuli at said frequency; or
(d) addition of more burst stimuli at said frequency.

9. A method of stimulating nerve tissue of a patient in need thereof by using a pulse generator of an implantable medical device, said method comprising the steps of:
(a) storing, in said implantable medical device, a first stimulation parameter that defines a frequency to be used as a lower bound of a frequency spectrum;
(b) storing, in said implantable medical device, a second stimulation parameter that defines a frequency to be used as an upper bound of a frequency spectrum;
(c) generating, by said pulse generator, a first noise stimulus that comprises a frequency spectrum bounded by said first stimulation parameter and said second stimulation parameter, wherein frequency and power of said frequency spectrum are inversely proportional;
(d) generating, by said pulse generator, a second noise stimulus that comprises a frequency spectrum bounded by said first stimulation parameter and said second stimulation parameter, wherein frequency and power of said frequency spectrum are directly proportional;
(e) delivering said stimuli from said pulse generator to at least one stimulation lead; and
(f) applying said stimuli to nerve tissue of said patient via at least one electrode of said at least one stimulation lead.

10. The method of claim 9, wherein said noise stimuli are further defined as having one or more frequency spectrums having a power spectral density per unit of bandwidth proportional to 1/f^{(-β)} noise and of one or more frequency spectrums having a power spectral density per unit of bandwidth proportional to 1/f^{β} noise, wherein f is signal frequency and wherein β is any real number but excludes 0.

11. The method of claim 9, wherein β=1.

12. The method of claim 9, wherein β=2.

13. The method of claim 9, wherein β>2.

14. The method of claim 9, wherein said noise stimulus has one or more frequency spectrums having a power spectral density per unit of bandwidth proportional to 1/f^{(-β)} noise and of one or more frequency spectrums having a power spectral density per unit of bandwidth proportional to 1/f^{β} noise, wherein f is signal frequency and wherein β is a value that excludes 0, and said stimulus is combined with at least one stimulation pulse to be repeated in a tonic manner.

15. The method of claim 9, wherein said stimulus has one or more frequency spectrums having a power spectral density per unit of bandwidth proportional to 1/f^{(-β)} noise and of one or more frequency spectrums having a power spectral density per unit of bandwidth proportional to 1/f^{β} noise, wherein f is signal frequency and wherein β is a value that excludes 0, and said stimulus is combined with a burst stimulus that comprises a plurality of groups of spike pulses, each individually charge balanced or charge balanced at the end of the group of monophasic spikes.

16. A method of stimulating nerve tissue of a patient in need thereof by using a pulse generator of an implantable medical device, said method comprising the steps of:
(a) storing, in said implantable medical device, a first stimulation parameter that defines a frequency to be used as a lower bound of a frequency spectrum;
(b) storing, in said implantable medical device, a second stimulation parameter that defines a frequency to be used as an upper bound of a frequency spectrum;
(c) storing, in said implantable medical device, a third stimulation parameter that defines a frequency peak of predetermined amplitude;
(d) generating, by said pulse generator, a first noise stimulus that comprises a frequency spectrum bounded by said first stimulation parameter and said second stimulation parameter, wherein frequency and power of said frequency spectrum are inversely proportional, and wherein a frequency peak of predetermined amplitude occurs at a frequency determined by said third stimulation parameter;
(e) generating, by said pulse generator, a second noise stimulus that comprises a frequency spectrum bounded by said first stimulation parameter and said second stimulation parameter, wherein frequency and power of said frequency spectrum are directly proportional, and wherein a frequency peak of predetermined amplitude occurs at a frequency determined by said third stimulation parameter;
(f) delivering said stimuli from said pulse generator to at least one stimulation lead; and
(g) applying said stimuli to nerve tissue of said patient via at least one electrode of said at least one stimulation lead.

17. The method of claim 16, wherein said noise stimulus has one or more frequency spectrums having a power spectral density per unit of bandwidth proportional to 1/f^{(-β)} noise and of one or more frequency spectrums having a power spectral density per unit of bandwidth proportional to 1/f^{β} noise, wherein f is signal frequency and wherein β is any real number but excludes 0, and said noise stimulus is combined with at least one pulse stimulus to be repeated in a tonic manner and with a burst stimulation that comprises a plurality of groups of spike pulses each individually charge balanced or charge balanced at the end of the group of monophasic spikes.

18. The method of claim 16, wherein said frequency peak occurs at a frequency between 0 and 4 Hertz.

19. The method of claim 16, wherein said frequency peak occurs at a frequency between 4 and 7 Hertz.

20. The method of claim 16, wherein said frequency peak occurs at a frequency between 8 and 12 Hertz.

21. The method of claim 16, wherein said frequency peak occurs at a frequency between 12 and 30 Hertz.
